(19) United States
(12) Patent Application Publication
(10) Pub. No.: US 2014/0343448 A1
(43) Pub. Date: Nov. 20, 2014

(54) TWO-ELECTRODE, IMPEDANCE-BASED RESPIRATION DETERMINATION

(71) Applicant: Zephyr Technology Corporation, Annapolis, MD (US)

(72) Inventors: BRIAN KEITH RUSSELL, Annapolis, MD (US); JONATHAN JAMES WOODWARD, Annapolis, MD (US); AMIT KUMAR MUKHERJEE, Elkridge, MD (US); DANIEL WAYNE BARTLETT, Annapolis, MD (US); CHRIS SOLOMON, Manukau (NZ)

(73) Assignee: Zephyr Technology Corporation, Annapolis, MD (US)

(21) Appl. No.: 14/279,003

(22) Filed: May 15, 2014

Related U.S. Application Data
(60) Provisional application No. 61/823,593, filed on May 15, 2013.

(51) Int. Cl. A61B 5/08

(52) U.S. Cl. CPC A61B 5/0816; A61B 5/0809
USPC 600/536

(57) ABSTRACT

Methods, apparatuses and systems are described for determining respiration through impedance measurements using only two electrodes. A drive signal may be applied to a person, using only two electrodes. Using the same electrodes, the fluctuations in the voltage of the drive signal are determined. The voltage fluctuations in the drive signal are the result of impedance variations in the person's thoracic cavity due to respiration. Therefore, the voltage fluctuations may be used to determine a respiration rate of the person. In doing so, the voltage fluctuations may be digitized using a sampling rate that is much less than the frequency of the applied drive signal.

### CROSS-REFERENCE TO RELATED APPLICATIONS

This application claims priority to U.S. Provisional Patent Application No. 61/823,593, filed on May 15, 2013, the entirety of which is incorporated by reference herein.

### TECHNICAL FIELD

The present disclosure relates generally to physiological monitoring systems, and more particularly to physiological monitoring systems for impedance-based respiration determination.

### BACKGROUND

Respiration rate can be determined by monitoring a person's thoracic impedance. As the person breathes, changes in the size and air content of the thorax cause small changes in conductivity. The change in conductivity associated with breathing can be measured by passing a drive signal (typically having a frequency of approximately 50 kHz) through the thorax and measuring changes in potential difference.

Thoracic impedance is typically measured using electrocardiogram-type (ECG-type) electrodes adhered to the person's skin. Electrode contact resistance, however, can be highly variable, transient, non-linear, and/or unpredictable. Accordingly, noise associated with contact resistance and/or other sources, can be several orders of magnitude greater than the signal associated with respiration. For example, contact resistance can vary suddenly and unpredictably by up to 300Ω or more due to changes in pressure on the electrode, impact associated with foot strikes, perspiration, changes in body posture, and/or many other factors. The signal change in impedance based on respiration, on the other hand, can be approximately 0.1-1 Ω/in. Thus, the signal-to-noise ratio for measuring respiration through thoracic impedance is very small.

Traditionally, impedance-based respiration measurements have used high-precision techniques, such as four-wire ohmic measurement, to extract the signal from the noise. Such techniques can include increasing the current injected into the person, increasing the distance between the measurement electrodes, and/or high-fidelity analog-to-digital signal processing. Such known techniques, however, can require increased power consumption (and commensurate decreased battery life), expensive precision hardware, and/or uncomfortable and/or unwieldy electrodes and associated wires running across the person's body. Alternatively, the noise may be minimized by carefully controlling the person's environment. While such laboratory settings may be suitable for a person at rest for a relatively short observation, it is not feasible to replicate the laboratory environment in the field to measure an active person engaged in a variety of activities, over an extended period of time.

Therefore, a need exists for an improved impedance-based respiration rate detection method, system and apparatus.

### SUMMARY

The described features generally relate to one or more improved methods, systems, or apparatuses for determining respiration through impedance measurements using only two electrodes. For example, a drive signal may be applied to a person, using only two electrodes. Using the same electrodes, the fluctuations in the voltage of the drive signal are determined. The voltage fluctuations in the drive signal are the result of impedance variations in the person's thoracic cavity due to respiration. Therefore, the voltage fluctuations may be used to determine a respiration rate of the person. In doing so, the voltage fluctuations may be digitized using a sampling rate that is much less than the frequency of the applied drive signal.

As a result of the present disclosure, an improved impedance-based respiration rate detection method, system and apparatus may be used. By using only two electrodes to both drive and sense an applied signal, the impedance-based system reduces bulk, weight and complexity. Battery life may be improved. Additionally, by determining the voltage fluctuations that result from respiration-induced impedance changes and by processing these detected voltage fluctuations so that the resulting waveform may be digitally sampled using a sampling rate that is less than the frequency of the initial drive signal, processing time and power consumption may also be reduced. The digitized signal may also be adaptively filtered using additional physiological or environmental data to improve the accuracy of the respiration rate determination.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

Further scope of the applicability of the described methods and apparatuses will become apparent from the following detailed description, claims, and drawings. The detailed description and specific examples are given by way of illustration only, since various changes and modifications within the spirit and scope of the description will become apparent to those skilled in the art.

### BRIEF DESCRIPTION OF THE DRAWINGS

A further understanding of the nature and advantages of the present invention may be realized by reference to the following drawings. In the appended figures, similar components or features may have the same reference label. Further, various components of the same type may be distinguished by following the reference label by a dash and a second label that distinguishes among the similar components. If only the first reference label is used in the specification, the description is applicable to any one of the similar components having the same first reference label irrespective of the second reference label.

FIG. 1 is a block diagram of an example of a remote physiological parameter monitoring system;

FIG. 2 is a circuit diagram of an example circuit for a two-electrode impedance-based determination of respiration rate, in accordance with various embodiments;

FIG. 3 is a block diagram of an example of a sensor apparatus in accordance with various embodiments;

FIG. 4 is a block diagram of an example of a sensor apparatus in accordance with various embodiments;

FIG. 5A is a block diagram of an example of a respiration determination module in accordance with various embodiments;

FIG. 5B is an illustration of example waveforms that may be used in determining a respiration rate, in accordance with various embodiments;

FIG. 6 is a block diagram of an example of a sensor device in accordance with various embodiments;

FIG. 7 is a block diagram of an example of a server for communicating with a remote sensor device; and

FIGS. 8 and 9 are flowcharts of various methods for determining a person's respiration rate, in accordance with various embodiments.

### DETAILED DESCRIPTION

Traditionally, impedance-based respiration measurements have used high-precision techniques, such as four-wire ohmic measurement, to extract the signal from the noise. Such techniques can include increasing the current injected into the person, increasing the distance between the measurement electrodes, and/or high-fidelity analog-to-digital signal processing. These techniques, however, can require increased power consumption (and commensurate decreased battery life), expensive precision hardware, and/or uncomfortable and/or unwieldy electrodes and associated wires running across the person's body. These disadvantages may be avoided, however, by using the disclosed methods, systems and devices that utilize only two electrodes. For example, a drive signal may be applied to a person. The drive signal may be applied using only two electrodes. Using the same electrodes, the fluctuations in the voltage of the drive signal are determined. The voltage fluctuations in the drive signal may be the result of impedance variations in the person's thoracic cavity due to respiration. Therefore, the voltage fluctuations may be used to determine a respiration rate of the person. In doing so, the voltage fluctuations may be digitized using a sampling rate that is much less than the frequency of the applied drive signal. Because the sampling rate (and resulting bandwidth) of the digitized signal is thus reduced, the power, time and other resources needed to process the digitized signal may also be reduced.

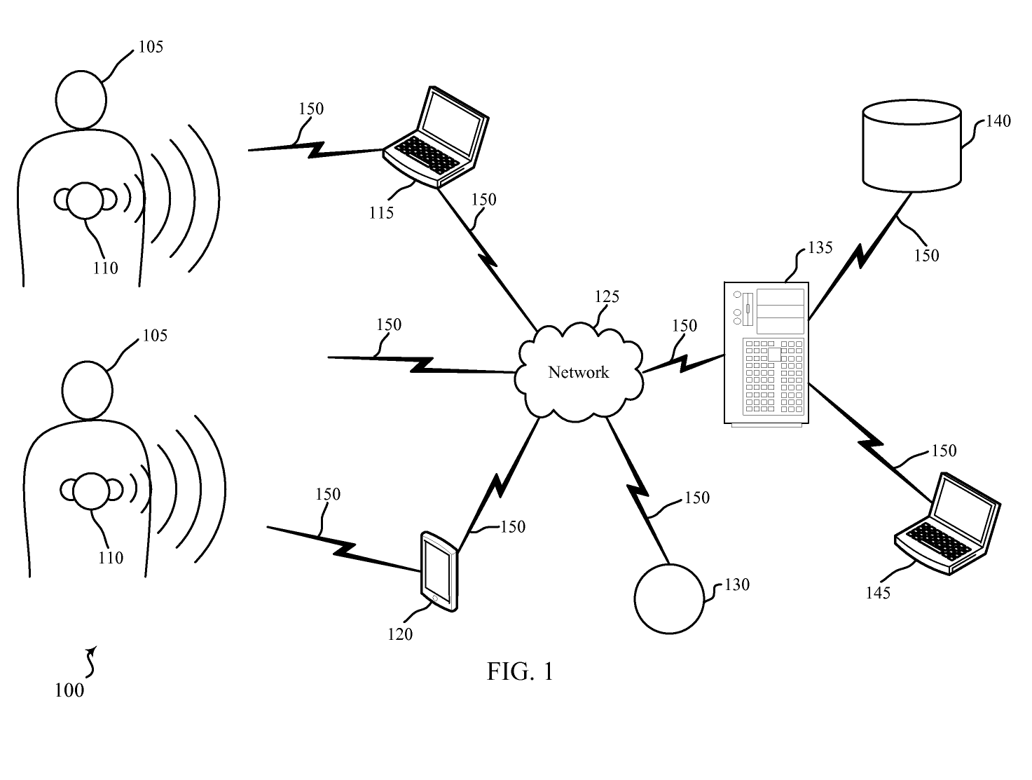

Referring first to FIG. 1, a diagram illustrates an example of a remote physiological parameter monitoring system 100. As an example, the system 100 may be a remote respiration rate monitoring system. The system 100 includes persons 105, each wearing a sensor unit 110. The sensor units 110 transmit signals via wireless communication links 150. The transmitted signals may be transmitted to local computing devices 115, 120. Local computer device 115 may be a local care-giver's station, for example. Local computer device 120 may be a mobile device, for example. The local computing devices 115, 120 may be in communication with a server 135 via network 125. The sensor units 110 may also communicate directly with the server 135 via the network 125. Additional, third-party sensors 130 may also communicate directly with the server 135 via the network 125. The server 135 may be in further communication with a remote computer device 145, thus allowing a care-giver to remotely monitor the persons 105. The server 135 may also be in communication with various medical databases 140 where the collected data may be stored.

The sensor units 110 are described in greater detail below. Each sensor unit 110, however, is capable of sensing multiple physiological parameters, including a person's respiration rate. However, the sensor units 110 may each include multiple sensors such as heart rate and ECG sensors, respiratory rate sensors, and accelerometers. For example, a first sensor in a sensor unit 110 can be a accelerometer operable to detect a user's posture and/or activity level. In such an embodiment, the first sensor can be operable to determine whether the user is standing, sitting, laying down, and/or engaged in physical activity, such as running A second sensor within a sensor unit 110 can be operable to detect a second physiological parameter. For example, the second sensor can be an electrocardiogram (ECG) sensing module, a breathing rate sensing module, and/or any other suitable module for monitoring any suitable physiological parameter. The data collected by the sensor units 110 may be wirelessly conveyed to either the local computer devices 115, 120 or to the remote computer device 145 (via the network 125 and server 135). Data transmission may occur via, for example, frequencies appropriate for a personal area network (such as Bluetooth or IR communications) or local or wide area network frequencies such as radio frequencies specified by the IEEE 802.15.4 standard.

The local computer devices 115, 120 may enable the person 105 and/or a local care-giver to monitor the collected physiological data. For example, the local computer devices 115, 120 may be operable to present data collected from sensor units 110 in a human-readable format. For example, the received data may be output as a display on a computer or a mobile device. The local computer devices 115, 120 may include a processor that may be operable to present data received from the sensor units 110, including alerts, in a visual format. The local computer devices 115, 120 may also output data and/or alerts in an audible format using, for example, a speaker.

The local computer devices 115, 120 can be custom computing entities configured to interact with the sensor units 110. In some embodiments, the local computer devices 115, 120 and the sensor units 110 may be portions of a single sensing unit operable to sense and display physiological parameters. In another embodiment, the local computer devices 115, 120 can be general purpose computing entities such as a personal computing device, such as a desktop computer, a laptop computer, a netbook, a tablet personal computer (PC), an iPod®, an iPad®, a smart phone (e.g., an iPhone®, an Android® phone, a Blackberry®, a Windows® phone, etc.), a mobile phone, a personal digital assistant (PDA), and/or any other suitable device operable to send and receive signals, store and retrieve data, and/or execute modules.

The local computer devices 115, 120 may include memory, a processor, an output, and a communication module. The processor can be a general purpose processor, a Field Programmable Gate Array (FPGA), an Application Specific Integrated Circuit (ASIC), a Digital Signal Processor (DSP), and/or the like. The processor can be configured to retrieve data from and/or write data to the memory. The memory can be, for example, a random access memory (RAM), a memory buffer, a hard drive, a database, an erasable programmable read only memory (EPROM), an electrically erasable programmable read only memory (EEPROM), a read only memory (ROM), a flash memory, a hard disk, a floppy disk, cloud storage, and/or so forth. In some embodiments, the local computer devices 115, 120 can include one or more hardware-based modules (e.g., DSP, FPGA, ASIC) and/or software-based modules (e.g., a module of computer code stored at the memory and executed at the processor, a set of processor-readable instructions that can be stored at the memory and executed at the processor) associated with executing an application, such as, for example, receiving and displaying data from sensor units 110.

The processor of the local computer devices 115, 120 can be operable to control operation of the output of the local computer devices 115, 120. The output can be a television, a liquid crystal display (LCD) monitor, a cathode ray tube (CRT) monitor, speaker, tactile output device, and/or the like. In some embodiments, the output can be an integral component of the local computer devices 115, 120. Similarly stated, the output can be directly coupled to the processor. For example, the output can be the integral display of a tablet and/or smart phone. In some embodiments, an output module can include, for example, a High Definition Multimedia Interface™ (HDMI) connector, a Video Graphics Array (VGA) connector, a Universal Serial Bus™ (USB) connector, a tip, ring, sleeve (TRS) connector, and/or any other suitable connector operable to couple the local computer devices 115, 120 to the output.

As described in additional detail herein, at least one of the sensor units 110 can be operable to transmit physiological data to the local computer devices 115, 120 and/or to the remote computer device 145 continuously, at scheduled intervals, when requested, and/or when certain conditions are satisfied (e.g., during an alarm condition). The transmitted physiological data may be respiration rate data.

The remote computer device 145 can be a computing entity operable to enable a remote user to monitor the output of the sensor units 110. The remote computer device 145 can be functionally and/or structurally similar to the local computer devices 115, 120 and can be operable to receive and/or send signals to at least one of the sensor units 110 via the network 125. The network 125 can be the Internet, an intranet, a personal area network, a local area network (LAN), a wide area network (WAN), a virtual network, a telecommunications network implemented as a wired network and/or wireless network, etc. The remote computer device 145 can receive and/or send signals over the network 125 via communication links 150.

The remote computer device 145 can be used by, for example, a health care professional to monitor the output of the sensor units 110. In some embodiments, as described in further detail herein, the remote computer device 145 can receive an indication of physiological data when the sensors detect an alert condition, when the healthcare provider requests the information, at scheduled intervals, and/or at the request of the healthcare provider and/or the person 105.

The server 135 may be configured to communicate with the sensor units 110, the local computer devices 115, 120, third-party sensors 130, the remote computer device 145 and databases 140. The server 135 may perform additional processing on signals received from the sensor units 110, local computer devices 115, 120 or third-party sensors 130, or may simply forward the received information to the remote computer device 145 and databases 140. The databases 140 may be examples of electronic health records (“EHRs”) and/or personal health records (“PHRs”), and may be provided by various service providers. The third-party sensor 130 may be a sensor that is not attached to the person 105 but that still provides data that may be useful in connection with the data provided by sensor units 110.

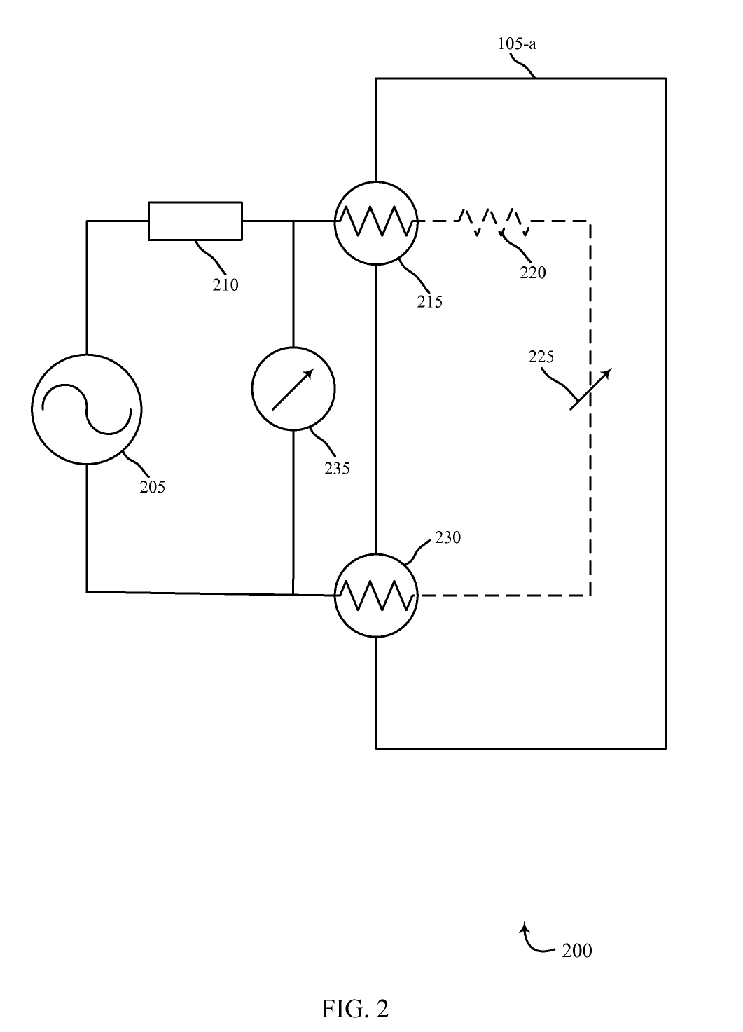

FIG. 2 is a schematic diagram of a two-electrode, impedance-based respiration sensing circuit 200 that may be included in one of the sensor units 110 of FIG. 1. The respiration sensing circuit 200 may include a signal source 205 coupled to a person 105-a via two electrodes 215, 230. The person 105-a may be an example of one of the persons 105 illustrated in FIG. 1. A detector 235 may be disposed parallel to the signal source 205 and may be operable to measure the impedance of person 105-a. The impedance of the person can include contact resistances associated with the electrodes 215, 230, a relatively constant thoracic impedance 220, and a variable thoracic impedance 225, which can change with respiration.

The signal source 205 can generate a drive signal suitable for injection into the person 105-a. The signal source 205 can generate a waveform having any suitable waveform, frequency, and/or current. For example, the signal source 205 can generate a 50 kHz square or sine wave. Additionally, the signal source 205 can generate either a fixed or variable frequency signal. As described in further detail herein, the characteristics of the waveform generated by the signal source 205 are not necessarily important for detection of the variable impedance 225 of the thorax associated with respiration. Accordingly, the signal source 205 can be operable to alter the characteristics of the waveform, for example, to avoid interference, to select a carrier suitable for some other physiological monitoring (e.g., dehydration), to tune the sensing circuit 200 to increase the sensitivity of the detector 235, etc. The signal source 205 can generate a drive signal having a frequency of approximately 20 kHz, 30 kHz, 50 kHz, 75 kHz, 100 kHz, and/or any other suitable frequency. In some embodiments, the signal source 205 can include wave shaping and/or protection circuitry, for example, to increase person safety.

A drive resistor 210 can be in series with and/or integral to the signal source 205. The drive resistor 210 can be operable to cause the person 105-a to be supplied a high-impedance signal and/or to isolate the signal generator 205 from feedback. In addition or alternatively, in some embodiments, the drive resistor 210 can be selected to be approximately equal the sum of the contact resistance associated with the electrodes 215, 230 and a steady state thoracic resistance 220. Similarly stated, the drive resistor 210 can be selected to impedance-match the signal generator 205 to the person 105-a, which can increase the sensitivity of the respiration sensing circuit 200 to changes in the impedance of the thorax 225. For example, in some embodiments the drive resistor 210 can have a resistance of approximately 2 kΩ, 4 kΩ, 6 kΩ, 10 kΩ, and/or any other suitable resistance. In some embodiments, the drive resistor 210 can be a variable resistor operable to be adjusted to be approximately equal to the sum of the contact resistance associated with the electrodes 215, 230, and a steady state thoracic resistance 220.

The electrodes 215, 230 can be ECG-type electrodes. In some embodiments, the electrodes 215, 230 can be commercially available. Similarly stated, in some embodiments, the signal generator 205 can be electrically coupled to the person 105-a via replaceable and/or disposable off-the-shelf ECG-type electrodes.

The electrodes 215, 230 electrically couple the signal generator 205 to the person 105-a, completing the sensing circuit 200. In some embodiments, the distance between the center points of the electrodes 215, 230 can be less than 7 inches, less than 5 inches, less than 2.5 inches, and/or any other suitable distance.

When activated, the signal generator 205 produces a waveform which is transmitted through the electrodes 215, 230 and the person 105-a. As the impedance of the thorax varies with respiration (e.g., as the variable impedance of the thorax 225 changes), the amplitude of the waveform produced by the signal generator 205, as measured at the person, is modulated.

The detector 235 can be coupled to the electrodes 215, 230, for example, in parallel with the series combination of the signal generator 205 and the drive resistor 210. As described in further detail herein, the detector 235 can be operable to measure the electric potential between the electrodes 215, 230, demodulate a signal associated with the electric potential between the electrodes 215, 230, calculate the variable impedance of the thorax 225 associated with respiration, calculate a respiration signal and/or rate, and/or store and/or transmit signals associated with respiration.

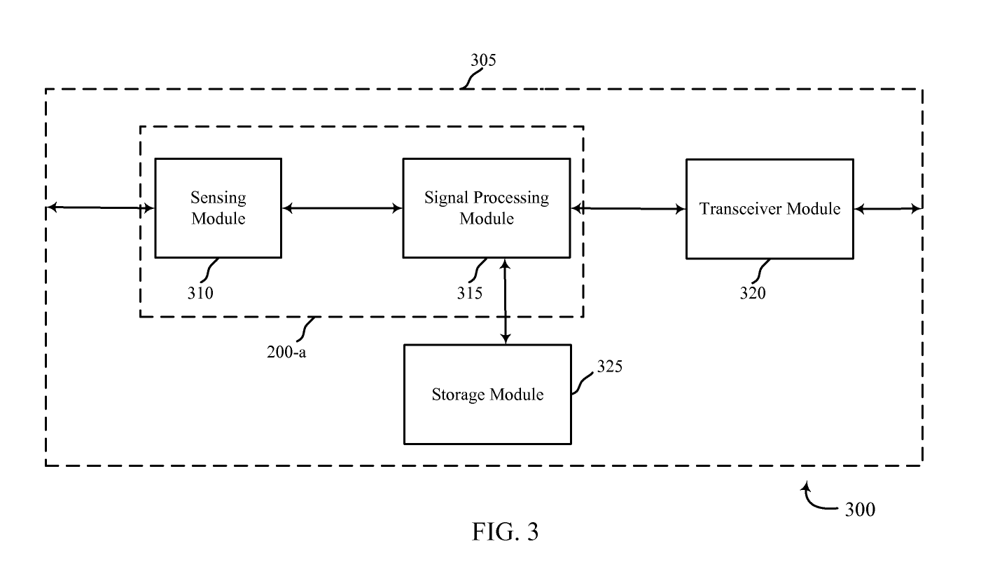

FIG. 3 is an example of a block diagram 300 of an apparatus 305 that may be used for sensing and determining a respiration rate, in accordance with various aspects of the present disclosure. In some examples, the apparatus 305 may be an example of aspects of one or more of the sensor units 110 described with reference to FIG. 1, and may sense, determine and transmit respiration rate information. The apparatus 305 may also be a processor. The apparatus 305 may include a sensing module 310, a signal processing module 315, or a transceiver module 320. Each of these components may be in communication with each other. As explained below, the sensing module 310 and the signal processing module 315 may correspond to aspects of the sensing circuit 200 of FIG. 2.

The components of the apparatus 305 may, individually or collectively, be implemented using one or more application-specific integrated circuits (ASICs) adapted to perform some or all of the applicable functions in hardware. Alternatively, the functions may be performed by one or more other processing units (or cores), on one or more integrated circuits. In other examples, other types of integrated circuits may be used (e.g., Structured/Platform ASICs, Field Programmable Gate Arrays (FPGAs), and other Semi-Custom ICs), which may be programmed in any manner known in the art. The functions of each unit may also be implemented, in whole or in part, with instructions embodied in a memory, formatted to be executed by one or more general or application-specific processors.

In some examples, the sensing module 310 may include at least one sensor. Alternatively, the apparatus 305 may include multiple sensing modules 310, each associated with at least one sensor. As an example, the sensing module 310 can include a respiration rate sensor. In addition, the sensing module 310 may include other sensors such as an accelerometer operable to detect a person's posture and/or activity level. Thus, the sensing module 310 may be operable to determine whether the person is standing, sitting, laying down, and/or engaged in physical activity, such as running. The sensing module 310 may further include an electrocardiogram (ECG) sensing module, a breathing rate sensing module, and/or any other suitable module for monitoring any suitable physiological parameter.

In some examples, the signal processing module 315 includes circuitry, logic, hardware and/or software for processing the signals output by the sensing module 310. The signal processing module 315 may include filters, analog-to-digital converters and other digital signal processing units. Data processed by the signal processing module 315 may be stored in a buffer, for example, in the storage module 325. The storage module 325 may include magnetic, optical or solid-state memory options for storing data processed by the signal processing module 315.

In some examples, the transceiver module 320 may be operable to send and/or receive signals between the sensor units 110 and either the local computer devices 115, 120 or the remote computer device 145 via the network 125 and server 135. The transceiver module 320 can include wired and/or wireless connectors. For example, in some embodiments, sensor units 110 can be portions of a wired or wireless sensor network, coupled by the transceiver module 320. The transceiver module 320 can be a wireless network interface controller (“NIC”), Bluetooth® controller, IR communication controller, ZigBee® controller and/or the like.

In some examples, the sensing module 310 and the signal processing module 315 may represent aspects of the sensing circuit 200 of FIG. 2. The sensing module 310 may correspond to, for example, the signal source 205 of circuit 200, while the signal processing module 315 may correspond to the detector 235 of circuit 200. Sensing module 310 and signal processing module 315 include additional logic and/or circuitry for managing the sensing and processing of a person's respiration rate, as described below.

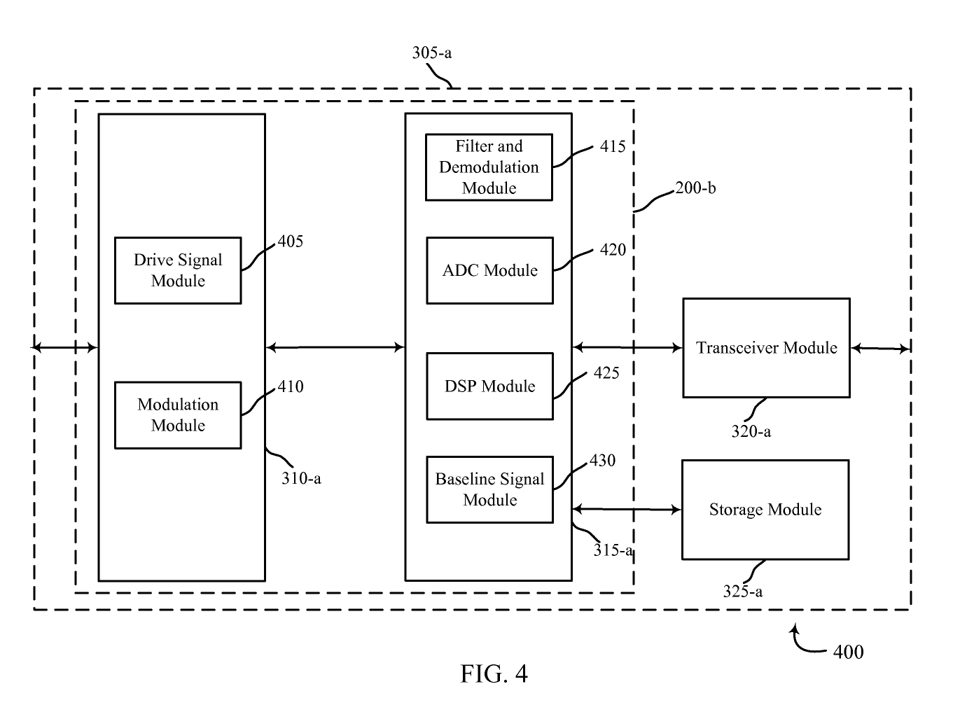

FIG. 4 shows a block diagram 400 that includes apparatus 305-a, which may be an example of one or more aspects of the apparatus 305 (of FIG. 3) for use in remote physiological monitoring, determining and transmitting of respiration rate signals, in accordance with various aspects of the present disclosure. In some examples, the apparatus 305-a may include a sensing module 310-a, a signal processing module 315-a, a storage module 325-a, and a transceiver module 320-a, which may be examples of the sensing module 310, the signal processing module 315, the storage module 325 and transceiver module 320 of FIG. 3. The sensing module 310-a and the signal processing module 315-a may represent aspects of sensing circuit 200-b, which may be an example of the sensing circuit 200 of FIG. 2. In some examples, the sensing module 310-a may include a drive signal module 405 and/or a modulation module 410. In additional examples, the signal processing module 315-a may include a filter and demodulation module 415, an analog-to-digital conversion (ADC) module 420, a digital signal processing (DSP) module 425, and/or a baseline signal module 430. The modules 405, 410, 415, 420, 425 and/or 430 may each be used in aspects of sensing and processing a person's respiration rate, as described below. While FIG. 4 illustrates a specific example, the functions performed by each of the modules 405, 410, 415, 420, 425 and/or 430 may be combined or implemented in one or more other modules.

The drive signal module 405 may be used to generate a drive signal suitable for application to a person. The drive signal module 405 can generate a waveform having any suitable waveform, frequency, and/or current. For example, the drive signal module 405 can generate a 50 kHz square or sine wave. Additionally, the signal source 205 can generate either a fixed or variable frequency signal. Other examples of frequencies that may be used in a generated drive signal include frequencies that are approximately 20 kHz, 30 kHz, 50 kHz, 75 kHz, 100 kHz, and/or any other suitable frequency. The generated drive signal is used to interrogate the variable impedance of a person's thoracic cavity.

In some embodiments, the modulation module 410 may be used to modulate the drive signal before application to the person. Modulation of the drive signal may include wave shaping, for example, to increase person safety. Additionally, the drive signal is also modulated as it passes through the person. For example, the drive signal may be modulated by variations in the impedance of the thorax, e.g., the variable impedance of the thorax 225 as shown and described with reference to FIG. 2. Thus, generating the drive signal via the drive signal module 405 can include generating a waveform using a current source, and modulation of the drive signal, at the modulation module 410, can include varying the impedance of a sensing circuit (e.g. the sensing circuit 200) such that the amplitude of the voltage of the waveform varies.

The filter and demodulation module 415 in the signal processing module 315-a can be used to filter and demodulate the drive signal after application to a person. For example, the sensed waveform can be filtered. The filtering can be analog filtering and can include a low-pass filter operable to attenuate noise associated with impacts (e.g., associated with foot strikes). The filter can also include a notch-filter to attenuate line-frequency interference and/or any other constant and/or predictable interfering frequency noise.

The filter and demodulation module 415 can also demodulate the sensed signal. Demodulation may involve using envelope detection. In this way, a relatively low-frequency signal associated with respiration, which typically does not contain frequency components above about 70 Hz, can be isolated from a relatively high-frequency drive signal, which can have a frequency within a range of approximately 25 kHz to 100 kHz. The envelope detection can be performed in the analog domain and can be carrier frequency-independent. Similarly stated, a demodulator can be tuned to the signal generator. The demodulator and the signal generator can be pre-set to operate at the same frequency, and/or the demodulator can be adjusted to the frequency produced by the signal generator by feedback control.

Another technique that may be used, though at the time of conversion to a digital signal, is synchronous detection. In a synchronous detection method, differences between the sensed signal and the drive signal are determined by digitizing the sensed signal by synchronizing an analog-to-digital sampling time with the drive signal so that the sensed signal is sampled at a same point in time during each period of the sensed waveform. As a result, the analog-to-digital conversion process acts as a mixer to produce a signal representing the voltage differences which also represents a low-frequency signal associated with respiration.

By demodulating in the analog domain, a relatively low-frequency signal can be presented to an analog to digital converter. Traditional methods for impedance-based respiration measurement detect absolute magnitude and phase of thoracic impedance in order to achieve a precision impedance measurement. In order to detect absolute magnitude and phase of impedance, the carrier signal is digitized for precision digital demodulation. In such a traditional embodiment the analog-to-digital converter would typically sample the voltage at a rate of at least twice the frequency of the signal generator to avoid aliasing. Because the signal generator typically operates at approximately 50 kHz, in traditional embodiments, analog to digital converters typically sample at least at 100 kHz, and normally at more than 1 MHz.

Thus, in signal processing module 315-a, the filter and demodulate module 415 demodulates the sensed signal and then passes the signal to the ADC module 420 for conversion to the digital realm. By using envelop detection before passing the signal to an analog-to-digital converter, the analog-to-digital converter can operate at or below 1 kHz. For example, the analog to digital conversion, performed by the ADC module 420, can be performed at 100 Hz, 40 Hz, 25 Hz, and/or any other suitable sample rate. Noise associated with impacts, heart movement, varying contact resistance, etc. can have frequency components that overlap the frequency range of the signal and/or can have a very low frequency component that can cause the signal to drift. Thus, the demodulated signal can have a large dynamic range that would saturate typical analog-to-digital converters and/or traditional noise reduction circuitry.

To provide for the dynamic range of the demodulated signal, the analog-to-digital conversion at the ADC module 420 can be performed by a high resolution analog-to-digital converter. For example, in some embodiments, the analog to digital conversion can be performed by a 20, 24, or 32 bit analog to digital conversion. By applying, for example, envelope detection before converting the signal into the digital domain, the data sampling rate can be decreased, which can allow for the use of cheaper, slower, and/or lower power electronics for digital signal processing, as described in further detail herein.

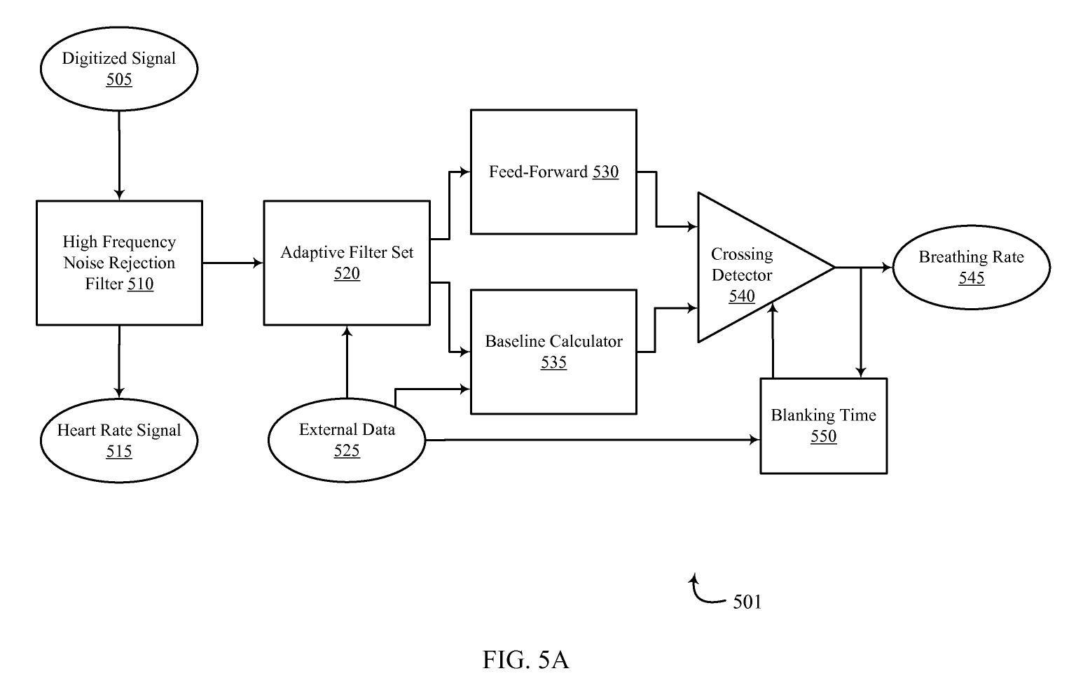

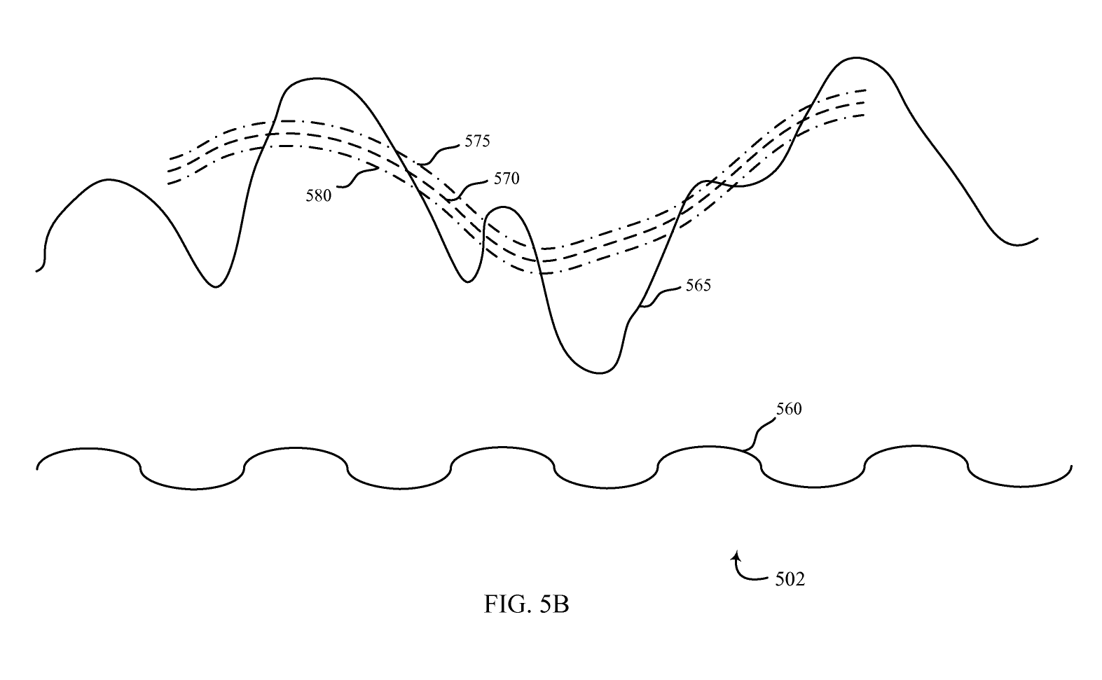

The DSP module 425 applies further processing to the digitized signal output by the ADC module 420. For example, the digitized signal output by the ADC module 420 may be further filtered to remove high frequency noise. An adaptive filter may additionally be used to further filter the digital signal based on external data, and as explained in greater detail with relation to FIG. 5A.

The baseline signal module 430 is operable to generate a baseline signal which may be used to calculate a respiration rate of a person. A respiration rate can be calculated by detecting the digitized impedance signal as it crosses a baseline. Thus, the baseline signal module 430 can calculate a moving average of the digitized signal. The length of the moving average window can be fixed or variable. In some embodiments, the length of the moving average window can correspond to the respiration rate. For example, the length of the moving average can approximate the wave period of the digitized signal, or 0.75 times the wave period, 1.25 times the wave period, 2 times the wave period, and/or any other suitable length. In some embodiments, it may be desirable to set the moving average window length as approximately a whole-number multiple of the respiration rate, such that a full-cycle average of the signal can be computed. Additional details related to the calculation of the baseline signal and an associated respiration rate are provided with respect to FIG. 5A.

FIG. 5A is a schematic diagram 501 of a digital signal processing method, according to an embodiment. Aspects of the digital signal processing diagram 501 correspond to the functions performed by the DSP module 425 and the baseline signal module 430 of FIG. 4. In particular, the high frequency noise rejection filter 510 and the adaptive filter set 520 of diagram 501 may correspond to the DSP module 425 of FIG. 4. The feed-forward module 530 and the baseline calculator 535, in addition to the crossing detector 540 and the blanking time module 550, may correspond to the baseline signal module 430 of FIG. 4.

In the diagram 501, a digitized signal 505 (representing, for example, the digitized signal output by the ADC module 420 of FIG. 4) is supplied to a high frequency noise rejection filter 510. The high frequency noise rejection filter 510 can be a median filter. A median filter can be particularly effective at eliminating impulse noise, for example, noise associated with heart movement. In some embodiments, a heart rate signal 515 can be obtained by comparing the output of the high frequency noise rejection filter 510 to the unfiltered digital signal 505.

An adaptive filter set 520 is operable to further filter the signal based on external data 525. The adaptive filter set 520 can include a band pass filter operable to selectively pass the frequency range associated with normal human respiration. The adaptive filter set 520 can be operable to restrict signal bandwidth to the frequency range of interest (e.g., the frequency range associated with respiration) and/or adjust the gain to improve detection sensitivity. Because respiratory patterns change with a number of factors including posture, activity level, etc., which may be difficult to infer from the digitized signal 505 itself, the adaptive filter set can be operable to receive external data 525, from a sensor such as an accelerometer.

Using an accelerometer, the adaptive filter set 520 can be operable to determine the person's body orientation and/or posture. For example, the adaptive filter set 520 can be operable to determine whether the person is laying down, standing, sitting, slouching, etc. The adaptive filter set 520 can also be able to determine activity level, for example, based on frequency of foot strikes, body motion, etc. In response, the adaptive filter set 520 can be operable to adjust the width of the pass band. For example, a person laying down and not moving can be presumed to be at rest. If the person is presumed to be at rest, the adaptive filter set 520 can select a filtering regime operable to pass a relatively large frequency range associated with at-rest respiration, such as a pass band from approximately 0.01 Hz to 10 Hz and apply a relatively large gain to magnify the signal. Similarly, when external data 525 indicates a high activity level that can be associated with running, the signal associated with respiration can be stronger, so the adaptive filter set 520 can be operable to pass a relatively narrower band; for example, the pass window can be approximately 0.5 Hz to 5 Hz.

Although described as accelerometer data, any external data 525 that can be correlated with respiration can be used by the adaptive filter set 520. For example, a global positioning sensor can be used to indicate whether the person is stationary, moving at a speed associated with walking, moving at a speed associated with running, or moving at a speed associated with traveling in a car. In other embodiments, atmospheric data, such as smog, pollen, atmospheric pressure, etc. can be correlated with respiration and used to adjust the parameters of the adaptive filter, particularly if the person is asthmatic, allergic, and/or has other respiratory issues. Person health data, such as inhaler use (e.g., from a “smart” inhaler), medical history, previous respiration data, information associated with apnea, etc. can be used to adjust the adaptive filter set 520 and, in some embodiments, be used to personalize a respiration sensing device for the person.

Because neither respiration nor impedance-related noise are deterministic, in some embodiments, adaptive filtering is more effective than pure frequency domain searching. For example, the use of Fourier-based methods to determine respiration rate can prove unsatisfactory since it may not be possible to determine whether a frequency response is due to respiration or noise. Because respiration, however, generally occurs within fairly predictable range of frequencies, especially if external indicia such as posture and activity are taken into account, the use of the adaptive filter set 520 can be an effective method for increasing the signal-to-noise ratio.

A breathing rate 545 can be calculated by detecting the crossing of a baseline signal by the digitized and filtered impedance signal. A baseline calculator 535 can be operable to generate a baseline signal by calculating a moving average of the digitized and filtered signal. The length of the moving average window can be fixed or variable. In some embodiments, the length of the moving average window can correspond to the respiration rate. For example, the length of the moving average can approximate the wave period of the signal 564, or 0.75 times the wave period, 1.25 times the wave period, 2 times the wave period, and/or any other suitable length. In some embodiments, the moving average window length may be set to be inversely proportional to the person's expected respiration rate. In some embodiments, it may be desirable to set the moving average window length as approximately a whole-number multiple of the respiration rate, such that a full-cycle average of the signal can be computed. Thus, in some embodiments, the breathing rate 545 can be fed-back to the baseline calculator 535 to set the moving average window length. Additionally, the baseline calculator 535 may be modified by external data 525.

Typically, moving average modules return a time-delayed response. Similarly stated, the output of a moving average module will typically lag the signal, returning an average for previously received data. In order to improve the accuracy of crossing-point detection, the baseline signal can be shifted forward in the time domain using the feed-forward module 530. The feed-forward module 530 can be operable to synchronize the phase of a determined baseline signal with the phase of the digitized and filtered signal output from the adaptive filter set 520. By using the feed-forward module 530, the length of the moving average window can be increased, which can result in a more stable baseline and a more accurate breathing rate 545.

The breathing rate 545 can be calculated using the crossing detector 540 to detect the rate at which the digitized and filtered signal output from the feed-forward module 530 crosses the baseline output by the baseline calculator 535. This is illustrated in the waveform diagram 502 of FIG. 5B. In FIG. 5B, a signal 565 (e.g., the output of the feed-forward module 530) is shown as it crosses a baseline 570 (e.g., the output of the baseline calculator 535). In diagram 502, a representation of actual respiration 560 is also illustrated. As can be illustrated, the breathing rate 545 can be half the rate at which the signal 565 crosses the baseline 570, where the signal 565 can have either a positive or a negative slope when crossing the baseline. For example, a full cycle of respiration can include crossing the baseline 570 once during inhalation and once during exhalation. Additionally, diagram 502 illustrates that the baseline 570 is flanked by a zone marked by boundaries 575 and 580. An even more robust method of determining respiration rate is to consider each time that the signal 565 enters the zone bounded by waveforms 575 and 580. In other words, the zone bounding the baseline 570 essentially widens the baseline so as to eliminate false crossings due to noise. FIG. 5B is illustrative, is not to scale, and does not represent experimental data.

Traditional methods of calculating a breathing rate 545 include detecting crossings of a static threshold (e.g., the midpoint of the dynamic range of the signal). Such a method, however, can cause signal loss when there is a transient input that shifts the signal from the previous baseline, as might be associated with a change of pressure on an electrode, signal drift, and/or noise having a frequency component similar to the breathing rate 545.

Returning to FIG. 5A, the blanking time module 550 can be operable to reject spurious crossings. The blanking time module 550 can be operable to reject all crossings (or entrances into the zone bounding the baseline 570, as illustrated in FIG. 5B) that occur within less than a specified blanking time. For example, the blanking time module 480 can reject crossings occurring in less than 1 s, less than 0.5 s, less than 0.2 s, and/or any other suitable time. The blanking time can be based on biological indications. For example, if it is unlikely that a specific individual will take two breaths or more within one second, the blanking time module 550 can reject a second baseline crossing within a one second blanking time as spurious.

In some embodiments, the blanking time can vary based on changes in actual and/or expected respiratory rate. For example, the blanking time module 550 can monitor the respiration rate (e.g., receive feedback) and set the blanking time as a fractional value of the breathing rate 545. For example, the blanking time can be ¼ the breathing rate 545, ⅛ the breathing rate, 1/12 the breathing rate, and/or any other suitable value. In another embodiment, the blanking time module 550 can be operable to adjust the blanking time based on the external data 525. For example, if an accelerometer indicates a change in posture or activity, the blanking time can be adjusted. For example, if the external data 525 indicates that the person has moved from a prone position to a standing position, the blanking time can be decreased. Similarly, if the external data 525 indicates the person has transitioned from standing to running, the blanking time can be decreased.

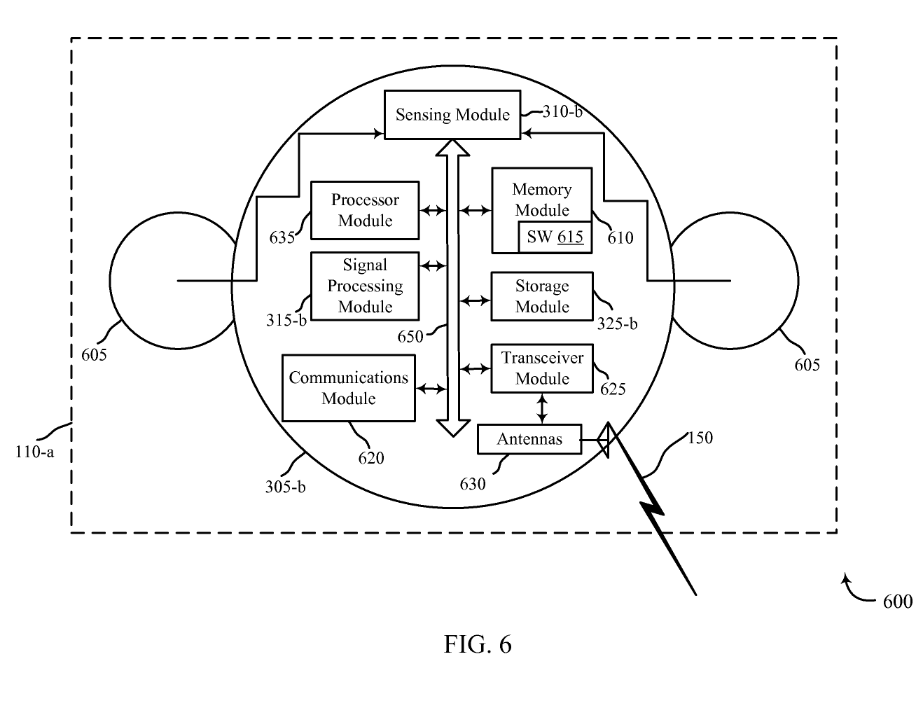

FIG. 6 shows a block diagram 600 of a sensor unit 110-a for use in remote monitoring and determination of a person's respiratory rate, in accordance with various aspects of the present disclosure. The sensor unit 110-a may have various configurations. The sensor unit 110-a may, in some examples, have an internal power supply (not shown), such as a small battery, to facilitate mobile operation. In some examples, the sensor unit 110-a may be an example of one or more aspects of one of the sensor units 110 and/or apparatus 305 described with reference to FIGS. 1, 3, 4 and/or 5A. The sensor unit 110-a may be configured to implement at least some of the features and functions described with reference to FIGS. 1, 2, 3, 4 and/or 5A.

The sensor unit 110-a may include one or more electrodes 605 and a sensing apparatus 305-b. The sensing apparatus 305-b may further include a sensing module 310-b, a processor module 635, a memory module 610, a communications module 620, at least one transceiver module 625, at least one antenna (represented by antennas 630), a storage module 325-b, or a signal processing module 315-b. Each of these components may be in communication with each other, directly or indirectly, over one or more buses 650. The sensing module 310-b, the storage module 325-b, and the signal processing module 315-b may be examples of the sensing module 310, the storage module 325, and the signal processing module 315, respectively, of FIGS. 3 and 4.

The memory module 610 may include random access memory (RAM) or read-only memory (ROM). The memory module 410 may store computer-readable, computer-executable software (SW) code 615 containing instructions that are configured to, when executed, cause the processor module 635 to perform various functions described herein for determining a respiration rate, for example. Alternatively, the software code 615 may not be directly executable by the processor module 635 but be configured to cause the sensor unit 110-a (e.g., when compiled and executed) to perform various of the functions described herein.

The processor module 635 may include an intelligent hardware device, e.g., a CPU, a microcontroller, an ASIC, etc. The processor module 635 may process information received through the transceiver module 625 or information to be sent to the transceiver module 625 for transmission through the antenna 630. The processor module 635 may handle, alone or in connection with the sensing module 310-b and the signal processing module 315-b, various aspects of signal processing as well as determining and transmitting a respiration rate.

The transceiver module 625 may include a modem configured to modulate packets and provide the modulated packets to the antennas 630 for transmission, and to demodulate packets received from the antennas 630. The transceiver module 625 may, in some examples, be implemented as one or more transmitter modules and one or more separate receiver modules. The transceiver module 625 may support transmission of a respiration rate. The transceiver module 625 may be configured to communicate bi-directionally, via the antennas 635 and communication link 150, with, for example, local computer devices 115, 120 and/or the remote computer device 145 (via network 125 and server 135 of FIG. 1). Communications through the transceiver module 625 may be coordinated, at least in part, by the communications module 620. While the sensor unit 110-a may include a single antenna, there may be examples in which the sensor unit 110-a may include multiple antennas 630.

The sensing module 310-b and the signal processing module 315-b may be configured to perform or control some or all of the features or functions described with reference to FIGS. 1, 2, 3, 4 and/or 5A related to determination of a respiration rate. For example, the sensing module 310-b may be configured to generate a drive signal for application to a person. The signal processing module 315-b may be configured to sense voltage fluctuations in the generated drive signal. The signal processing module 315-b may be further configured to filter and demodulate the sensed voltage fluctuations. The signal processing module 315-b may digitize the sensed voltage fluctuations after the fluctuations have been demodulated. Using additional digital signal processing, the signal processing module 315-b may be configured to determine a baseline signal from the digitized signal, and using these signals, determine a respiration rate of a person. The sensing module 310-b and the signal processing module 315-b, or portions of these modules, may include a processor, or some or all of the functions of the sensing module 310-b and the signal processing module 315-b may be performed by the processor module 635 or in connection with the processor module 635. Additionally, the sensing module 310-b and the signal processing module 315-b, or portions of these modules, may include a memory, or some or all of the functions of the sensing module 310-b and the signal processing module 315-b may use the memory module 610 or be used in connection with the memory module 610.

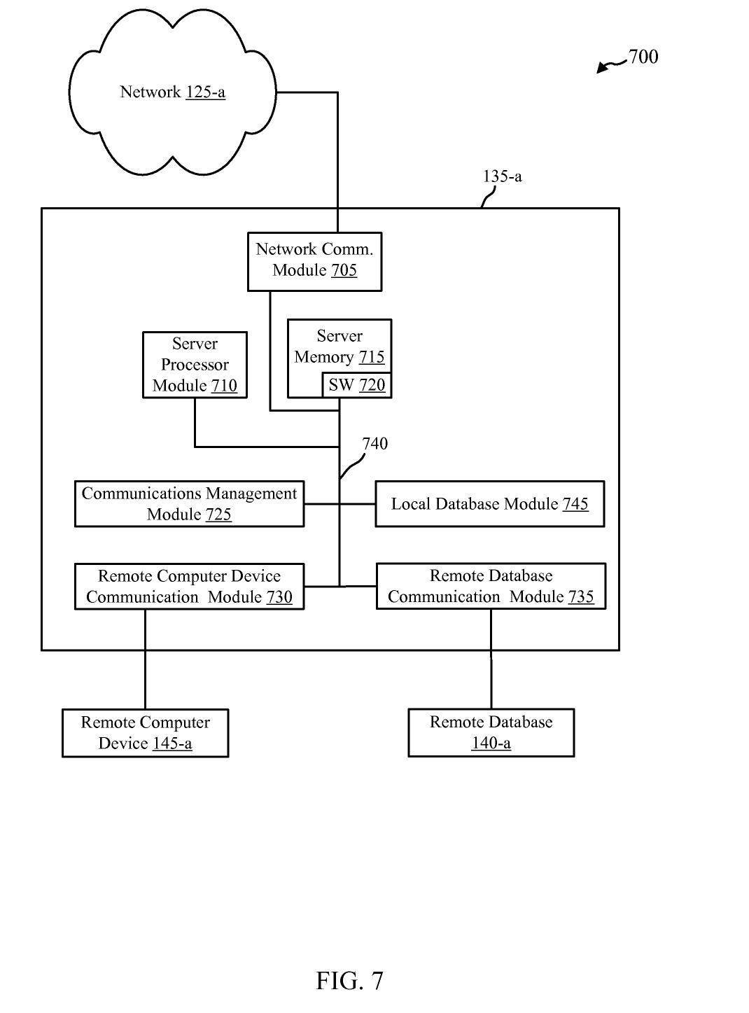

FIG. 7 shows a block diagram 700 of a server 135-a for use in remote determination of a person's respiratory rate, in accordance with various aspects of the present disclosure. In some examples, the server 135-a may be an example of aspects of the server 135 described with reference to FIG. 1. The server 135-a may be configured to implement or facilitate at least some of the server features and functions described with reference to FIG. 1.

The server 135-a may include a server processor module 710, a server memory module 715, a local database module 745, and/or a communications management module 725. The server 135-a may also include one or more of a network communication module 705, a remote computer device communication module 730, and/or a remote database communication module 735. Each of these components may be in communication with each other, directly or indirectly, over one or more buses 740.

The server memory module 715 may include RAM and/or ROM. The server memory module 715 may store computer-readable, computer-executable code 720 containing instructions that are configured to, when executed, cause the server processor module 710 to perform various functions described herein related to remote physiological monitoring. Alternatively, the code 720 may not be directly executable by the server processor module 710 but be configured to cause the server 135-a (e.g., when compiled and executed) to perform various of the functions described herein.

The server processor module 710 may include an intelligent hardware device, e.g., a central processing unit (CPU), a microcontroller, an ASIC, etc. The server processor module 710 may process information received through the one or more communication modules 705, 730, 735. The server processor module 710 may also process information to be sent to the one or more communication modules 705, 730, 735 for transmission. Communications received at or transmitted from the network communication module 705 may be received from or transmitted to sensor units 110, local computer devices 115, 120, or third-party sensors 130 via network 125-a, which may be an example of the network 125 described in relation to FIG. 1. Communications received at or transmitted from the remote computer device communication module 730 may be received from or transmitted to remote computer device 145-a, which may be an example of the remote computer device 145 described in relation to FIG. 1. Communications received at or transmitted from the remote database communication module 735 may be received from or transmitted to remote database 140-a, which may be an example of the remote database 125 described in relation to FIG. 1. Additionally, a local database may be accessed and stored at the server 135-a. The local database module 745 is used to access and manage the local database, which may include data received from the sensor units 110, the local computer devices 115, 120, the remote computer devices 145 or the third-party sensors 130 (of FIG. 1).

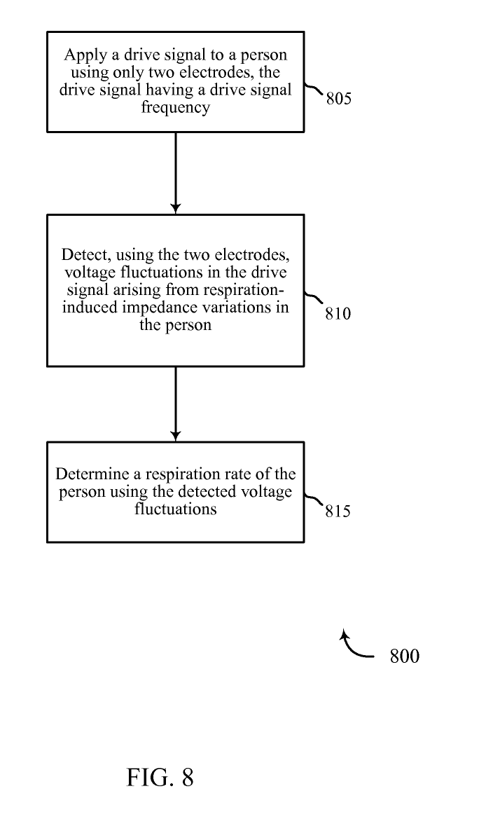

FIG. 8 is a flow chart illustrating an example of a method 800 for determining a respiration rate of a person, in accordance with various aspects of the present disclosure. For clarity, the method 800 is described below with reference to aspects of one or more of the sensor units 110 described with reference to FIGS. 1 and/or 6, respectively, or aspects of one or more of the apparatus 305 described with reference to FIGS. 3 and/or 4. In some examples, a sensor unit such as one of the sensor units 110 or an apparatus such as one of the apparatuses 305 may execute one or more sets of codes to control the functional elements of the sensor unit or apparatus to perform the functions described below.

At block 805, the method 800 may include applying a drive signal to a person using only two electrodes, the drive signal having a drive signal frequency. The drive signal may be applied by, for example, the sensing module 310 of FIGS. 3, 4 and/or 6.

At block 810, the method 800 may include detecting, using the two electrodes, voltage fluctuations in the drive signal arising from respiration-induced impedance variations in the person. The same electrodes used for applying the drive signal are used for the detecting of the voltage fluctuations. The detection may be performed by, for example, the signal processing module 315 of FIGS. 3, 4 and/or 6.

At block 815, the method 800 may include determining a respiration rate of the person using the detected voltage fluctuations. For example, the detected voltage fluctuations may be filtered in the analog domain, demodulated, digitized, and further filtered and processed in order to determine a baseline signal from which the person's respiration rate may be determined, as explained in connection with the signal processing module 315 of FIGS. 3, 4 and/or 6, including the description of diagram 501 of FIG. 5A.

It should be noted that the method 800 is just one implementation and that the operations of the method 800 may be rearranged or otherwise modified such that other implementations are possible.

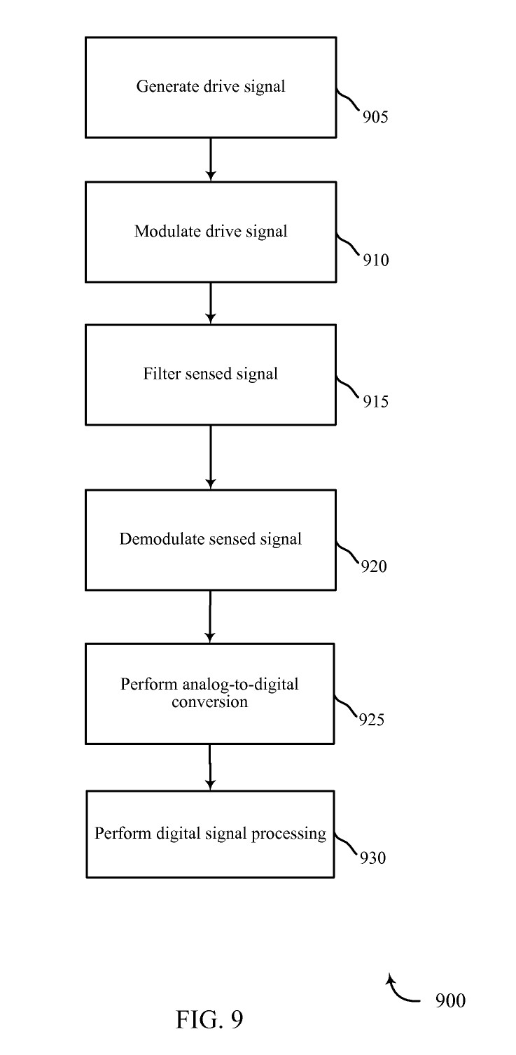

FIG. 9 is a flow chart illustrating an example of a method 900 for determining a respiration rate of a person, in accordance with various aspects of the present disclosure. For clarity, the method 900 is described below with reference to aspects of one or more of the sensor units 110 described with reference to FIGS. 1 and/or 6, respectively, or aspects of one or more of the apparatus 305 described with reference to FIGS. 3 and/or 4. In some examples, a sensor unit such as one of the sensor units 110 or an apparatus such as one of the apparatuses 305 may execute one or more sets of codes to control the functional elements of the sensor unit or apparatus to perform the functions described below.

At block 905 of method 900, a drive signal is generated and, at block 910, the drive signal is modulated. The drive signal can be generated, at block 905, by a signal generator, e.g., the signal generator 205 as shown and described with reference to FIG. 2, also represented by the sensing module 310, described with reference to FIGS. 3, 4 and/or 6. The drive signal can be modulated, at block 910, by variations in the impedance of the thorax, e.g., the variable impedance of the thorax 225 as shown and described with reference to FIG. 2. For example, generating the drive signal, at block 905, can include generating a waveform using a current source, and modulation of the drive signal, at block 910, can include varying the impedance of a sensing circuit (e.g. the sensing circuit 200 of FIG. 2) such that the amplitude of the voltage of the waveform varies.

The voltage of the waveform can be sensed by a detector (e.g. the detector 235 of FIG. 2, also described in connection with the signal processing module 315 of FIGS. 3, 4 and/or 6). The waveform can be filtered, at block 915. The filtering, at block 915, can be analog filtering and can include a low-pass filter operable to attenuate noise associated with impacts (e.g., associated with foot strikes). A low-pass filter can have with a cutoff frequency of 60 kHz, 75 kHz, 100 kHz, and/or any other suitable cutoff frequency operable to pass the drive signal. The filtering, at block 915, can also include a notch-filter to attenuate line-frequency interference (e.g. 50 and/or 60 Hz noise) and/or any other constant and/or predictable interfering frequency noise. In addition, the gain and offset of the waveform can be adjusted in the analog domain, at block 915.

At block 920, the detector can demodulate the signal using, for example, envelope detection. In this way, a relatively low-frequency signal associated with respiration, which typically does not contain frequency components above about 70 Hz, can be isolated from a relatively high-frequency drive signal, which can have a frequency within a range of approximately 25 kHz to 100 kHz. The envelope detection can be performed in the analog domain and can be carrier frequency independent. Similarly stated, a demodulator can be tuned to the signal generator. The demodulator and the signal generator can be pre-set to operate at the same frequency, and/or the demodulator can be adjusted to the frequency produced by the signal generator by feedback control.

By demodulating in the analog domain, at block 920, a relatively low-frequency signal can be presented to an analog-to-digital converter, at block 925. Traditional methods for impedance based respiration measurement detect absolute magnitude and phase of thoracic impedance in order to achieve a precision impedance measurement. In order to detect absolute magnitude and phase of impedance, the carrier signal is digitized for precision digital demodulation. In such a traditional embodiment the analog-to-digital converter would typically sample the voltage at a rate of at least twice the frequency of the signal generator to avoid aliasing. Because the signal generator typically operates at approximately 50 kHz, in traditional embodiments, analog-to-digital converters typically sample at least at 100 kHz, and normally at more than 1 MHz.

By using envelop detection, at block 920, before passing the signal to an analog-to-digital converter, at block 925, the analog-to-digital converter can operate at or below 1 kHz. For example, the analog-to-digital conversion, at block 925, can be performed at 100 Hz, 40 Hz, 25 Hz, and/or any other suitable sample rate. Noise associated with impacts, heart movement, varying contact resistance, etc. can have frequency components that overlap the frequency range of the signal and/or can have a very low frequency component that can cause the signal to drift. Thus, the demodulated signal can have a large dynamic range that would saturate typical analog to digital converters and/or traditional noise reduction circuitry.

To provide for the dynamic range of the demodulated signal, the analog-to-digital conversion, at block 925, can be performed by a high resolution analog-to-digital converter.

For example, in some embodiments, the analog-to-digital conversion can be performed by a 20-, 24-, or 32-bit analog-to-digital conversion. The available clock rate, size, cost, and/or power consumption render high resolution analog-to-digital converters unsuitable for synchronous detection applied in traditional impedance based respiration measurement used to determine the absolute magnitude and phase of thoracic impedance. By applying envelope detection, at block 920, before converting the signal into the digital domain, at block 925, the data sampling rate can be decreased, which can allow for the use of cheaper, slower, and/or lower power electronics for digital signal processing, at block 930, as described with relation to FIGS. 5A and 5B.

In addition, by applying envelope detection, at block 920, before analog-to-digital conversion, at block 925, the data acquisition and/or digital signal processing, at block 930, can be decoupled from the drive signal frequency. Similarly stated, the analog-to-digital sampling rate and/or the clock rate associated with digital signal processing can be selected based on the data signal, e.g., respiration rate, rather than excitation frequency. Furthermore, the drive signal and/or analog-to-digital sampling rate can be adjusted without requiring the digital signal processing clock rate to be adjusted.

It should be noted that the method 900 is just one implementation and that the operations of the method 900 may be rearranged or otherwise modified such that other implementations are possible.

While various embodiments have been described above, it should be understood that they have been presented by way of example only, and not limitation. Although various embodiments have been described as having particular features and/or combinations of components, other embodiments are possible having a combination of any features and/or components from any of embodiments as discussed above.

Where schematics and/or embodiments described above indicate certain components arranged in certain orientations or positions, the arrangement of components may be modified. While the embodiments have been particularly shown and described, it will be understood that various changes in form and details may be made.

The above description provides examples, and is not limiting of the scope, applicability, or configuration set forth in the claims. Changes may be made in the function and arrangement of elements discussed without departing from the spirit and scope of the disclosure. Various embodiments may omit, substitute, or add various procedures or components as appropriate. For instance, the methods described may be performed in an order different from that described, and various steps may be added, omitted, or combined. Also, features described with respect to certain embodiments may be combined in other embodiments.

The detailed description set forth above in connection with the appended drawings describes exemplary embodiments and does not represent the only embodiments that may be implemented or that are within the scope of the claims. The term “exemplary” used throughout this description means “serving as an example, instance, or illustration,” and not “preferred” or “advantageous over other embodiments.” The detailed description includes specific details for the purpose of providing an understanding of the described techniques. These techniques, however, may be practiced without these specific details. In some instances, well-known structures and devices are shown in block diagram form in order to avoid obscuring the concepts of the described embodiments.

Information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices, e.g., a combination of a DSP and a microprocessor, multiple microprocessors, one or more microprocessors in conjunction with a DSP core, or any other such configuration. A processor may in some cases be in electronic communication with a memory, where the memory stores instructions that are executable by the processor.

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope and spirit of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items indicates a disjunctive list such that, for example, a list of “at least one of A, B, or C” means A or B or C or AB or AC or BC or ABC (i.e., A and B and C).

A computer program product or computer-readable medium both include a computer-readable storage medium and communication medium, including any mediums that facilitates transfer of a computer program from one place to another. A storage medium may be any medium that can be accessed by a general purpose or special purpose computer. By way of example, and not limitation, computer-readable medium can comprise RAM, ROM, EEPROM, CD-ROM or other optical disk storage, magnetic disk storage or other magnetic storage devices, or any other medium that can be used to carry or store desired computer-readable program code in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote light source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include compact disc (CD), laser disc, optical disc, digital versatile disc (DVD), floppy disk and blu-ray disc where disks usually reproduce data magnetically, while discs reproduce data optically with lasers. Combinations of the above are also included within the scope of computer-readable media.

The previous description of the disclosure is provided to enable a person skilled in the art to make or use the disclosure. Various modifications to the disclosure will be readily apparent to those skilled in the art, and the generic principles defined herein may be applied to other variations without departing from the spirit or scope of the disclosure. Throughout this disclosure the term “example” or “exemplary” indicates an example or instance and does not imply or require any preference for the noted example. Thus, the disclosure is not to be limited to the examples and designs described herein but is to be accorded the widest scope consistent with the principles and novel features disclosed herein.

1. A method of determining respiration, comprising:
applying a drive signal to a person using only two electrodes, the drive signal having a drive signal frequency;
detecting, using the two electrodes, voltage fluctuations in the drive signal arising from respiration-induced impedance variations in the person; and
determining a respiration rate of the person using the detected voltage fluctuations.

2. The method of claim 1, further comprising:
modulating the drive signal applied to the person.

3. The method of claim 2, wherein the detecting of the voltage fluctuations in the drive signal comprises:
filtering the detected voltage fluctuations; and
demodulating the filtered voltage fluctuations.

4. The method of claim 3, further comprising:
using envelope detection to demodulate the filtered voltage fluctuations.

5. The method of claim 4, further comprising:
digitizing the demodulated filtered voltage fluctuations using an analog-to-digital converter and a sampling frequency that is less than the drive signal frequency.

6. The method of claim 5, wherein the sampling frequency is less than 1 kHz.

7. The method of claim 1, where the detecting of the voltage fluctuations in the drive signal comprises:
determining a difference between the drive signal and a sensed signal returned from the person as a result of the drive signal being applied to the person.

8. The method of claim 1, wherein applying the drive signal to the person further comprises:
using a non-ideal current source.

9. The method of claim 1, wherein the detecting of the voltage fluctuations in the drive signal comprises:
applying one or more adaptive filters, wherein a pass band of the one or more adaptive filters is modified based on external data representing one or more factors that influence respiration rates.

10. The method of claim 9, wherein the external data represents a posture or activity level of the person.

11. The method of claim 1, wherein the determining the respiration rate of the person comprises:
digitizing the detected voltage fluctuations; and
using an adaptive-length buffer to store the digitized voltage fluctuations as a baseline signal.

12. The method of claim 11, wherein a length of the adaptive length buffer is inversely proportional to an approximate respiration rate of the person.

13. The method of claim 11, further comprising comparing the baseline signal with a delayed representation of the digitized voltage fluctuations.

14. The method of claim 11, wherein the determining of the respiration rate further comprises:
determining a frequency by which the digitized voltage fluctuations crosses the baseline signal or enters a zone bounding the baseline signal.

15. The method of claim 14, further comprising:
using a blanking period to reject, in determining the frequency by which the digitized voltage fluctuations crosses the baseline signal or enters the zone bounding the baseline signal, one or more crossings or zone entrances that are within the blanking period.

16. The method of claim 15, further comprising:
modifying the blanking period based on an activity level of the person or other environmental or physiological inputs.

17. An impedance-based respiration determination device, comprising:
a signal generator for applying a drive signal to a person using only two electrodes, the drive signal having a drive signal frequency; and
at least one processor configured to:
detect, using the two electrodes, voltage fluctuations in the drive signal arising from respiration-induced impedance variations in the person; and
determine a respiration rate of the person using the detected voltage fluctuations.

18. The device of claim 17, further comprising:
one or more adaptive filters configured to filter the detected voltage fluctuations, wherein a pass band of the one or more adaptive filters is modified based on external data representing one or more factors that influence respiration rates.

19. A computer program product, comprising:
a non-transitory computer-readable medium having non-transitory program code recorded thereon, the non-transitory program code comprising:
program code to apply a drive signal to a person using only two electrodes, the drive signal having a drive signal frequency;
program code to detect voltage fluctuations in the drive signal arising from respiration-induced impedance variations in the person; and
program code to determine a respiration rate of the person using the detected voltage fluctuations.

20. The computer program product of claim 19, wherein the program code to detect the voltage fluctuations in the drive signal comprises:
program code to apply one or more adaptive filters, wherein a pass band of the one or more adaptive filters is modified based on external data representing one or more factors that influence respiration rates.
